(19) United States
(12) Patent Application Publication
(10) Pub. No.: US 2024/0216866 A1
(43) Pub. Date: Jul. 4, 2024

(54) DEVICE AND METHOD FOR BUNDLING HOLLOW FIBRE MEMBRANES

(71) Applicant: FRESENIUS MEDICAL CARE DEUTSCHLAND GMBH, Bad Homburg (DE)

(72) Inventors: Paul GASTAUER, Saarbruecken (DE); Franz KUGELMANN, St. Wendel (DE); Michael PAUL, Lebach (DE); Andreas RUFFING, Bexbach (DE); Tobias VEIT, Oberthal (DE)

(73) Assignee: FRESENIUS MEDICAL CARE DEUTSCHLAND GMBH, Bad Homburg (DE)

(21) Appl. No.: 18/286,561

(22) PCT Filed: Apr. 21, 2022
(86) PCT No.: PCT/EP2022/060603

§ 371 Date: Oct. 12, 2023

(30) Foreign Application Priority Data
Apr. 22, 2021 (DE) 10 2021 110 264.2

(51) Int. Cl. B01D 63/02

(52) U.S. Cl. CPC B01D 63/0233; B01D 63/0223

(57) ABSTRACT

The invention relates to a device and a method for producing a hollow fibre membrane bundle consisting of hollow fibre membranes, wherein, in the claimed device, a sheaf of hollow fibre membranes is received in a lower semitubular shell and bundled together to form a hollow fibre membrane bundle by a complementary upper semitubular shell.

The present invention relates to an apparatus and an arrangement for bundling hollow fiber membranes, and a method for bundling hollow fiber membranes using said apparatus.

### BACKGROUND

Hollow fiber membrane filters are used in the purification of liquids. In particular, hollow fiber membrane filters are used in medical technology for the purification and decontamination of water and in the therapy of kidney-damaged patients in extracorporeal blood treatment as dialyzers or hemofilters. The hollow fiber membrane filters generally consist of a cylindrical housing and a plurality of hollow fiber membranes arranged therein, which are cast at the ends in the housing with a casting compound in a casting zone and are sealingly connected to the housing. It is well known that such hollow fiber membrane filters are often designed in such a way that they are operated in the countercurrent process of two liquids, so that a particularly efficient mass transfer can take place via the membrane wall of the hollow fiber membranes and a desired purification of one of the liquids takes place. For this purpose, the hollow fiber membrane filters are designed in such a way that the lumens of the hollow fiber membranes form a first flow space through which a first liquid flows, and the interstices between the hollow fiber membranes in the housing of the hollow fiber membrane filter form a second flow space through which a second liquid can flow. Inlet or outlet chambers are provided at the end parts of the hollow fiber membrane filters, and the inlet or outlet chambers include fluid inlets to allow the first and second fluids to flow into and out of the respective flow spaces of the hollow fiber membrane filters.

The manufacture of such hollow fiber membrane filters is first preceded by the manufacture of the hollow fiber membranes. Hollow fiber membranes are manufactured in a spinning process. In the predominantly used processes, a spinning mass is provided which consists of a polymer solution having a solvent and polymers dissolved therein, e.g., polysulfone and polyvinylpyrrolidone. The spinning mass is extruded through an annular gap die to form a spun filament, which is introduced into a precipitation bath and precipitated to form the hollow fiber membrane. The resulting hollow fiber membrane is passed through further rinsing baths and drying zones and is assembled on a reel to form an array of hollow fiber membranes. For the manufacturing of the hollow fiber membrane filters, the reel of coiled hollow fiber membranes is bundled and cut into predetermined lengths. Typically, the bundles are made using envelope films, which are placed around the array of hollow fiber membranes. The hollow fiber membranes are thereby compressed in the envelope of the films and can thus be used as hollow fiber membrane bundles in the further production process. The envelope films are polymer films, such as polyethylene or PTFE films, or low-friction coated films, in particular PTFE- or polyolefin-coated special films. The cover films are placed and fixed around the hollow fiber membranes by folding techniques and/or welding processes. In the process, the hollow fiber membrane bundle automatically assumes a cylindrical shape.

In a further step, the hollow fiber membrane bundles wrapped in the films are inserted into the cylindrical housings of the hollow fiber membrane filters. The envelope foil is then pulled out of the housing again, while at the same time the hollow fiber membranes are held in place by a suitable tool against the pull of the envelope foil and remain in the housing of the hollow fiber membrane filter. Usually, the outer diameter of the hollow fiber membrane bundle wrapped in the envelope foil is smaller than the inner diameter of the cylindrical housing. It is particularly important that the hollow fiber membrane bundle is compressed by the wrapping foil. The hollow fiber membrane bundle is thus stiffened and can thus be inserted into the cylindrical housing. As the envelope foil is pulled out of the cylindrical housing, the hollow fiber membrane bundle conforms to the inner diameter of the cylindrical housing.

Further steps follow during the manufacturing process in which the ends of the hollow fiber membranes are closed by melting or by applying a pre-casting. The hollow fiber membranes are then cast in the cylindrical housing at the end regions and fixed in the housing. After the potting has cured, the lumens of the hollow fiber membranes are exposed again by cutting through the potting compounds at the ends. In a further step, end caps with fluid connections are placed on the cylindrical housing so that the first and second flow chambers and the inlet or outlet chambers are formed. Subsequently, the hollow fiber membrane filter produced in this way is sterilized and subjected to further process steps, e.g., leak testing.

DE 20 2017 104 293 U1 describes an apparatus for manufacturing a hollow fiber membrane filter, comprising, inter alia, an apparatus for inserting a hollow fiber membrane bundle into a housing of a hollow fiber dialyzer and an apparatus for sealing the ends of the hollow fiber membranes, wherein the apparatus for inserting a hollow fiber membrane bundle into the housing comprises a gripper and a pressure mechanism and the apparatus for sealing the ends of the hollow fiber membranes comprises a source of electromagnetic waves.

EP 3 600 630 B1 describes the production of hollow fiber membrane bundles using magnetically sealable envelope films.

WO 2018/178124 A1 describes a winding wheel for producing hollow fiber membrane bundles. The winding wheel has a plurality of apparatus, each having a lower part with a semi-cylindrical trough for receiving an envelope film and a plurality of hollow fiber membranes, and flaps attached to the lower parts and movably supported by hinges, the flaps having a cylinder segment shape in the direction toward the semi-cylindrical trough. The hollow fiber membranes are wrapped in the cover film by closing the flaps and cut between the respective apparatus to form respective cut-to-length hollow fiber membrane bundles.

A disadvantage of the methods described in the prior art for bundling hollow fiber membranes is the use of envelope films. In an automated manufacturing process, the use of envelope films requires the use of elaborate equipment on the machine side or requires that certain process steps be performed manually. Furthermore, the use of the specially coated envelope films in large-scale processes also represents a significant cost factor. In addition, the use of the envelope films requires the provision of individual process steps, such as wrapping the hollow fiber membranes in the film or sealing the envelope film, which complicate the process of bundling and manufacturing the hollow fiber membrane filters in terms of process technology.

The task underlying the invention was therefore to further improve the production of hollow fiber membrane filters in terms of process technology and cost reduction by optimized bundling of the hollow fiber membranes.

### SUMMARY OF THE INVENTION

In a first aspect, the task is solved by an apparatus having the features of claim 1. The features of claims 2 to 11 describe preferred embodiments.

In a second aspect, the task is solved by an arrangement having the features of claim 12. The features of claims 13 and 14 describe preferred embodiments.

In a third aspect, the task is solved by a method having the features of claim 15 using an apparatus having the features of the first aspect. The features of claims 16 to 19 describe preferred embodiments.

In a fourth aspect, the given task is solved by a use of an apparatus according to the features of claims 1 to 11 or an arrangement according to the features of claims 12 to 14 in the manufacture of a hollow fiber membrane filter.

### DESCRIPTION OF THE INVENTION

In a first aspect, the invention relates to an apparatus for bundling hollow fiber membranes, comprising a lower part comprising a lower tube half-shell having two side edges and an inner side comprising a concavely curved surface for receiving an array of hollow fiber membranes, an upper part comprising an upper tube half-shell complementary to the lower tube half-shell and having two side edges and an inner side comprising a concavely curved surface, wherein at least one of the lower part and the upper part are arranged movably relative to each other in the apparatus, and wherein the apparatus is configured such that the lower part and the upper part are positioned in a first position such that the lower tube half-shell can receive an array of hollow fiber membranes in the first position, and the lower part and the upper part are positioned in a second position such that the lower tube half-shell and the upper tube half-shell enclose a cavity such that an array of hollow fiber membranes present in the cavity can be bundled together.

The apparatus has the advantage that, in the second position of the lower part and the upper part, an array of hollow fiber membranes is bundled through the cavity formed by the lower and the upper tube half-shells and can be fed to further process steps as an array of hollow fiber membranes. In particular, the use of a cover film can be dispensed with, since the necessary compression for generating the hollow fiber membrane bundle is already generated via the lower and upper tube half-shells. Therefore, in the production of hollow fiber membrane filters, there is no need for equipment on the machine side that would otherwise have to be provided for wrapping and sealing the envelope film around the hollow fiber membrane bundle.

In this context, the terms “lower part and upper part” describe two interacting components in the function of the apparatus according to the invention. In a preferred embodiment, the lower part and the upper part may be arranged such that the lower part is closer to the earth's center of gravity. Alternatively, however, the lower part and the upper part may be in a different position relative to each other in the apparatus, for example, such that the lower part and the upper part are equally close to the earth's center of gravity, or such that the lower part is further away from the earth's center of gravity than the upper part.

In the context of the present application, the term “tube half-shell” refers to a half-shell describing a tube segment formed by a longitudinal cut of a tube. Tube half-shells may have a contour approximating a circular segment in a cross-section transverse to the longitudinal orientation. The term “half” in tube half-shell does not necessarily mean that the tube half-shells have the shape of a segment of an exact half of a tube. In particular, the lower tube half-shell may also be a tube segment that represents more than half of a tube, or the upper tube half-shell may be a tube segment that represents more than half of a tube. The tube half-shells have concavely curved surfaces. In the context of the present application, the term “concave” is to be understood to mean a surface in which a straight line between arbitrarily selectable points on that surface extends completely outside the tube half-shell. In particular, the lower and upper tube half-shells are worked out complementary to each other. The term “complementary” in this context means that the lower tube half-shell and the upper tube half-shell, when the lower part and the upper part are in the second position, form a tube cavity which serves to concentrate an array of hollow fiber membranes and place them in a compressed state. The term “compressed” in this context means that the array of hollow fiber membranes is compressed and bundled to a spatial dimension by the application of a force, and the bundle thus produced develops a restoring force.

In a first position, the upper part and the lower part are spaced apart from each other, that is, the inner sides of the tube half-shells are accessible and, for example, an array of hollow fiber membranes can be inserted into the lower tube half-shell. The apparatus described herein may, for example, be part of a reel onto which the hollow fiber membranes are wound, wherein the winding process inserts an array of hollow fiber membranes into the lower tube half-shell. Alternatively, the array of hollow fiber membranes may be inserted into the apparatus described herein in the form of one or more strands of hollow fiber membranes. The term “strand” as used herein refers to a plurality of hollow fiber membranes oriented in a uniform preferred direction relative to one another. In this context, the term “array of hollow fiber membranes” refers to a plurality of hollow fiber membranes consisting, for example, of one or more coiled strands. In particular, the term “array” also refers to the number of hollow fiber membranes that are combined into a bundle.

In the second position, the lower part and the upper part are positioned relative to each other such that the lower tube half-shell and the upper tube half-shell enclose a cavity. In particular, the lower part and the upper part may be in engagement with each other in the second position. In this context, the term “engaged” means that the lower part and the upper part are adjacent to each other such that the lower tube half-shell and the upper tube half-shell enclose a cavity. In one embodiment, the engagement may be accomplished by the lower part having a receiving area for the upper part in which the upper part positively connects. By relative movement of the lower part and upper part in the apparatus with respect to each other, the upper part is inserted into the receiving area of the lower part and the cavity is enclosed by the lower and upper tube half shells. In an alternative embodiment, the engagement can also be accomplished by the upper part having a receiving area for the lower part in which the lower part positively connects. By moving the lower part and upper part relative to each other in the apparatus, the lower part is inserted into the receiving area of the upper part and the cavity is enclosed by the lower and upper tube half-shells.

In one embodiment, the apparatus is characterized in that the concavely curved surface of the lower tube half-shell describes a segment of a substantially cylindrical shape; the concavely curved surface of the upper tube half-shell describes a segment of a substantially cylindrical shape such that the concavely curved surfaces of the lower and upper tube half-shells enclose a substantially cylindrical cavity in a second position of the lower part and the upper part.

Via the cylindrical cavity formed by the lower tube half-shell and the upper tube half-shell, the hollow fiber membrane bundle enclosed in the cavity takes on a cylindrical shape and can thus be advantageously used for constructing hollow fiber membrane filters having a cylindrical housing.

In a further embodiment, the apparatus is characterized in that the lower tube half shell is oversized relative to the upper tube half shell in the region of the side edges of the lower tube half shell, or in that the upper tube half shell is oversized relative to the lower tube half shell in the region of the side edges of the upper tube half shell, wherein the apparatus is configured such that in the second position of the lower part and the upper part, the upper tube half-shell is in engagement with the lower tube half-shell, or that in the second position of the lower part and the upper part, the lower tube half-shell is in engagement with the upper tube half-shell.

In the described embodiment, it is achieved that in the second position of the upper part and the lower part, one tube half-shell can engage in the area of the oversize of the other tube half-shell. It is thus possible to further compress the array of hollow fiber membranes in the enclosed cavity between the upper and lower tube half-shells. The term “compress” in this context means that the hollow fiber membrane bundle can be further compressed in the cavity.

In a further embodiment, the apparatus is characterized in that the side edges of the upper tube half-shell and/or the lower tube half-shell are chamfered. In particular, the chamfer strips hollow fiber membranes that are adjacent to the surface adjacent to the side edges of a respective tube half-shell, effectively displacing the membranes within an area within an intended compaction diameter. As a result, greater compaction of the hollow fiber membrane bundle between the upper and lower tube half-shells is achieved. For example, the chamfers on the side edges of the upper tube half-shell cause hollow fiber membranes abutting the surface of the lower tube half-shell adjacent to the side edges of the lower tube half-shell to be displaced into the lower shell within the range of an intended compaction diameter.

In another embodiment, the apparatus is characterized in that the concavely curved surfaces of the lower and/or upper tube half-shells include a plurality of drillings, and the apparatus is configured to flow or exhaust air through the drillings to the inside of the lower and/or upper tube half-shells.

The drillings may extend from a ventilation duct located in the lower part and/or upper part of the apparatus. In one embodiment, the lower part has at least two gas ports connected to the ventilation channel. Via the gas ports, the ventilation channel and the plurality of drillings, an air flow can be supplied to the inner side of the respective tube half-shells, thus supporting the bundling of the hollow fiber membranes and the further processing of the hollow fiber membrane bundle. In particular, the supplied air flow forms an air cushion between the hollow fiber membrane bundle and the surface of the lower and/or upper tube half-shell and reduces the friction of the hollow fiber membranes on the surfaces of the tube half-shells. The airflow assists in guiding a hollow fiber membrane bundle out of the cavity formed by the upper and lower tube half-shells, so that the risk of damage to the hollow fiber membranes is reduced.

In a further embodiment, the apparatus is characterized in that at least some of the drillings, preferably all of the drillings in the lower and/or upper tube half-shell are aligned in a preferred direction, in particular abutting at a coinciding angle of 10° to 80°, or 20° to 70°, or 30° to 60° to the central axis of the cylindrical cavity.

In the arrangement of the aligned drillings in a preferred direction, a flow direction of the inflowing air is created on the inner side of the lower and/or upper tube half-shell. In particular, the direction of the air flow on the inner sides of the tube half-shells assists in guiding the hollow fiber membrane bundle out of the cavity of the lower and upper tube half-shells in the direction of the flow direction of the air flow.

In a further embodiment, the apparatus is characterized in that the concavely curved surfaces of the lower and/or upper tube half-shells are provided with a coating. The coatings thereby reduce the friction between the surface and the hollow fiber membranes abutting thereon. In particular, the coatings can be plastic coatings, e.g. coatings of PTFE (polytetrafluoroethylene) or other fluorinated polymers with a low coefficient of friction. Alternatively, the surfaces may be coated with polyolefins. In another alternative embodiment, the surfaces may also be coated with a low friction ceramic coating or a DLC (diamond-like carbon) coating.

In a further embodiment, the apparatus is characterized in that the apparatus comprises at least one movable cutting device for cutting the hollow fiber membrane bundle in the second position of the upper part and the lower part to a predetermined length dimension. Advantageously, at least two cutting devices may be movably arranged in the apparatus. In this case, the apparatus is configured in such a way that the hollow fiber membranes protruding from the cavity of the lower and upper tube half shells in the second position of the lower part and upper part are cut. This brings the hollow fiber membrane bundle to a length intended for insertion of the hollow fiber membrane bundle into the housing of a hollow fiber membrane filter. The cutting device may include a mechanical cutting tool, such as a blade.

Alternatively, the cutting device may comprise a thermal cutting tool, that is, in particular, a cutting tool in which the ends of the hollow fiber membranes are cut off by melting using a hot wire, a hot blade, or a laser beam. Preferably, the melting of the ends of the hollow fiber membranes is done in such a way that the lumens of the hollow fiber membranes are closed. This has the advantage that the process step of pre-molding can be omitted in the manufacturing process of the hollow fiber membrane filters. At the same time, the hollow fiber membrane bundle is mechanically stabilized by the melting of the hollow fiber membranes in the end region. The hollow fiber membrane bundle can thus be handled more safely in the further manufacturing process of the hollow fiber membrane filter.

In a further embodiment, the apparatus is characterized in that the apparatus has a receiving unit for a housing tube, which is arranged movably in the apparatus relative to the lower part and/or the upper part, and the apparatus is further configured in such a way that, in the second position of the upper part and lower part, a housing tube can be arranged adjoining the lower and upper tube half-shells on the face side via a position of the receiving unit. Here, the relative movement of the receiving unit to the upper and/or lower part is decisive, i.e. it can also be provided that the receiving unit is configured to be stationary and the relative movement is carried out via the upper and/or lower shell. The hollow fiber membrane bundle can thus be inserted directly from the cavity of the lower tube half-shell and the upper tube half-shell into a subsequently arranged housing tube. The subsequently arranged housing tube is preferably the cylindrical housing of a hollow fiber membrane filter. In a further embodiment, the apparatus is characterized in that the apparatus comprises means for inserting the hollow fiber membrane bundle from the cavity into the end-connected housing tube. Preferably, the insertion of the hollow fiber membrane bundle into the adjoining housing tube is performed by a movable ejector with which the hollow fiber membrane bundle can be displaced from the cavity comprising the lower and upper tube half-shells into the subsequently arranged housing tube.

Preferably, the diameter of the cavity of lower and upper tube half-shell in the second position is smaller than the diameter of the housing tube by at least 2%, preferably at least 5%, more preferably at least 7%. Due to the smaller diameter, sliding the hollow fiber membrane bundle into the housing tube is particularly simplified. In a particular embodiment, the housing tube has a tapered central part. This means that the inner diameter of the center part of the housing tube has a smaller diameter than the inner diameters at the ends of the housing tube. Preferably, in such embodiments of the housing tube, the inner diameter decreases from the ends to the central part. Such a housing tube or hollow fiber membrane filter housing is identified by the term “tapered design” or “tapered center tube”. In this case, it is provided that the end of the housing tube, which in the second position of the upper part and lower part is arranged adjacent to the lower and upper tube half-shells on the face side, has a diameter at least 2%, preferably at least 5%, further preferably at least 7% larger than the diameter of the cavity of the lower and upper tube half-shells in the second position.

In a second aspect, the invention relates to an arrangement comprising an apparatus according to the first aspect of the invention and a hollow fiber membrane bundle located in the apparatus in the cavity formed by lower and upper tube half shells. In this regard, neither the hollow fiber bundle nor the apparatus comprises an envelope film. In one embodiment, the arrangement further comprises a housing tube disposed in the second position of the upper and lower parts in end-to-end relationship with the lower and upper tube half-shells, wherein the diameter of the hollow fiber bundle disposed in the cavity is at least 2%, preferably at least 5%, more preferably at least 7% less than the diameter of the housing tube. In another embodiment, the arrangement further comprises a housing tube having a tapered central part, wherein the diameter of the hollow fiber bundle located in the cavity is at least 2%, preferably at least 5%, more preferably at least 7% less than the diameter of the end of the housing tube located end-to-end with the lower and upper tube half-shells in the second position of the upper part and lower part.

In a third aspect, the invention relates to a method of bundling hollow fiber membranes, comprising the steps of: providing an apparatus according to at least one of the embodiments of the first aspect of the invention, placing an array of hollow fiber membranes in the lower tube half-shell in the first position of the upper and lower parts of the apparatus, relatively moving the upper and lower parts to the second position such that the array of hollow fiber membranes are bundled into a hollow fiber membrane bundle in a cavity formed from the lower and upper tube half-shells. Preferably, the hollow fiber membrane bundle is also compressed in the process. According to the process according to the invention, bundling of the hollow fiber membranes is carried out without using an envelope film. The hollow fiber membrane bundle present in the cavity is thereby preferably already compressed to a dimension that can be used for further use in the manufacture of a hollow fiber membrane filter. In one embodiment, the hollow fiber membrane bundle is compressed to a packing density of greater than 60%, in particular greater than 64%, further in particular greater than 66%. In the context of the present application, “packing density” is understood to be the proportion in the cavity, formed by the lower and upper tube half-shells, that is occupied by the hollow fiber membranes. The packing density is calculated from the percentage ratio of the sum of the cross-sectional areas of the hollow fiber membranes to the cross-sectional area of the cavity formed by the lower and upper tube half-shells.

In a further embodiment, the method is characterized in that the hollow fiber membrane bundle is lengthened to a predetermined length dimension in the second position of the upper part and the lower part using a cutting device. In a further embodiment, the method is further characterized in that the cutting device is a hot cutting tool that melts the ends of the hollow fiber membranes during cutting to length and seals the lumens of the hollow fiber membranes. The length of the hollow fiber membrane bundle is thus adjusted to a dimension necessary for further manufacturing of the hollow fiber membrane filter.

In another embodiment, the method is characterized by positioning a cylindrical housing tube adjacent to the cavity formed by lower and upper tube half-shells and sliding the hollow fiber membrane bundle into the adjacent cylindrical housing tube. The hollow fiber membrane bundle is pushed out of the cavity by an ejector that is pushed into the cavity, thereby displacing the hollow fiber membrane bundle toward the adjacent housing tube. The housing tube is in particular a cylindrical tube, in particular the cylindrical housing of a hollow fiber membrane filter. It is preferably provided that the hollow fiber bundle in the cavity formed by the lower and upper tube half-shells is compressed by at least 2%, preferably at least 5%, more preferably at least 7% more than a hollow fiber bundle inserted into the housing tube. Due to the stronger compression, the insertion of the hollow fiber membrane bundle into the housing tube is particularly simplified. If the housing tube has a tapered central part, it is provided that the hollow fiber bundle in the cavity formed by the lower and upper tube half-shell is compressed by at least 2%, preferably at least 5%, more preferably at least 7% more than a hollow fiber bundle at the end of the housing tube, which in the second position of the upper part and lower part of the apparatus is arranged adjacent to the lower and upper tube half-shell at the end face.

In a further embodiment, the method is characterized in that air flows against the inner surface of the lower and/or upper tube half-shell through the plurality of drillings. By flowing air against the inner side, an air cushion is created between the surface of the lower and/or upper tube half-shell and the hollow fiber membranes abutting the surface of the tube half-shells. Preferably, at least some of the drillings or all of the drillings are oriented in a preferential direction so that the flow of air against the inner surfaces of the tube half-shells adopts a preferential flow direction. This particularly assists the process of pushing the hollow fiber membrane bundle out of the apparatus. The pushing out of the hollow fiber membrane bundle thus occurs in the direction of the preferential flow direction of the approaching air.

In a fourth aspect, the invention relates to the use of an apparatus or arrangement according to at least one embodiment of the first or second aspect of the invention for manufacturing a hollow fiber membrane filter.

### DETAILED DESCRIPTION OF THE INVENTION BASED ON THE DRAWINGS

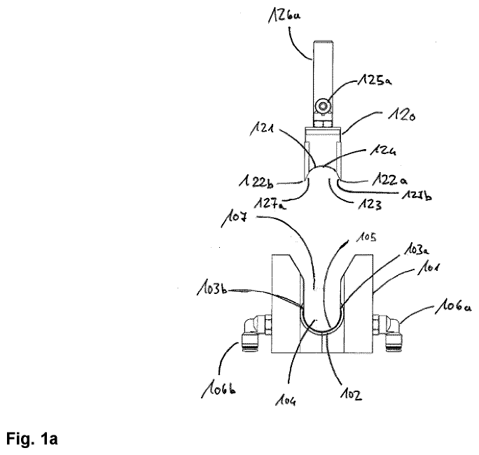

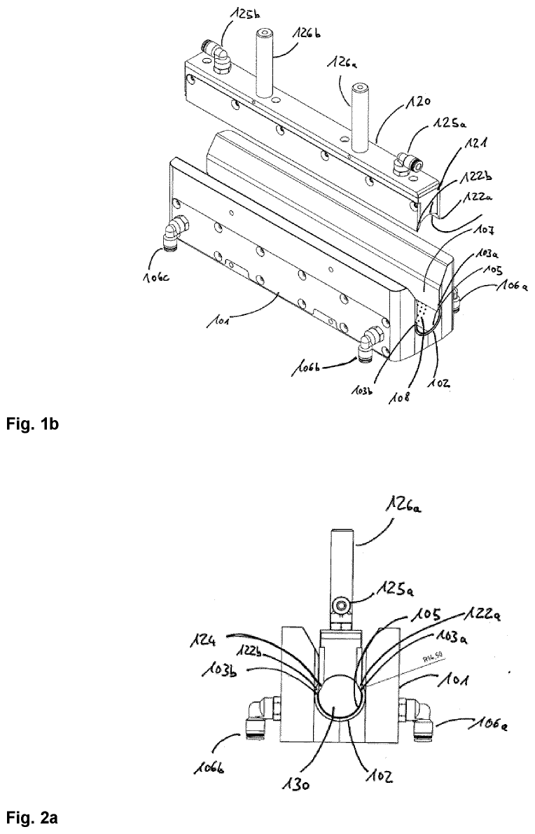

FIG. 1a shows in schematic view a cross-sectional view of the lower part 101 and the upper part 120. The cross-section shown is transverse to the longitudinal orientation of the lower part and the upper part. Not shown in FIG. 1a are further details of the apparatus, the components of which are the lower part and the upper part. FIG. 1a shows the lower part and the upper part in a first position in which the lower part and the upper part are spaced apart. Shown is an illustration in which the upper part is arranged above the lower part. However, other arrangements of the lower and upper parts in the first position are possible. Alternatively, the upper part may be arranged adjacent to the lower part in the first position. In the embodiment shown in FIG. 1a, the lower tube half-shell 102 is shown in a cross-sectional view. The cross-section of the lower tube half-shell is circular-segment shaped between side edges 103a and 103b, which are only visible in schematic cross-section in FIG. 1a. The lower tube half-shell has an inner surface 104 designed to receive an array of hollow fiber membranes, which are not shown in FIG. 1a. Furthermore, gas ports 106a and 106b are visible on the lower part 101, via which a gas flow, in particular an air flow, can be introduced into and removed from a ventilation duct (not shown in FIG. 1a) inside the lower part 101. The concavely curved surface of the lower tube half-shell is designated 105 in FIG. 1a. It is only visible as a segment of a circle in cross-section in FIG. 1a. In the embodiment shown, the lower part 101 further comprises a receiving area 107 for receiving the upper part 120.

The upper part 120 includes an upper tube half-shell 121 complementary to the lower tube half-shell 101. In the embodiment shown, the cross-section of the upper tube half-shell is circular-segment shaped in one section. The concavely curved surface of the upper tube half-shell is designated 124 in FIG. 1a. The side edges 122a and 122b, are only schematically visible in cross-section in FIG. 1a. The chamfers of the side edges are labeled 127a and 127b. Also shown are a retaining element 126a, by means of which the upper part is retained in the apparatus, and a gas connection 125a, by means of which a gas, in particular air, can be introduced into and discharged from a ventilation duct in the interior of the upper part 120 and flowed through drillings (not shown in FIG. 1a) onto the inner side 123 of the upper tube section.

FIG. 1b shows a schematic view of an oblique side view of lower part 101 and upper part 120 in the first position. Further shown with respect to FIG. 1a are a second retaining member 126b used to retain the upper section 120 in the apparatus, a second gas port 125b on the upper section and a third gas port 106c on the lower section, and drillings 108 in the surface 105 of the lower tube half shell 102.

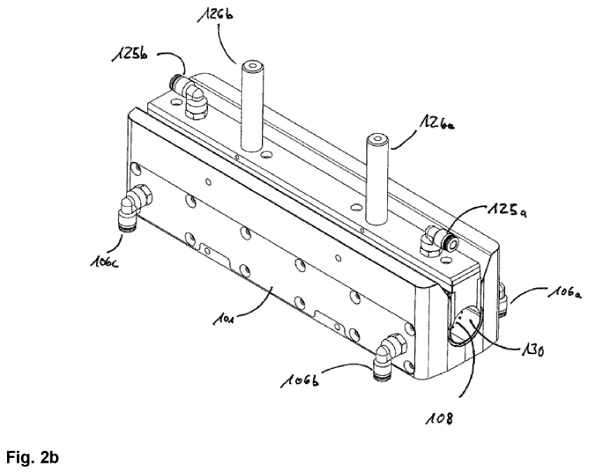

FIG. 2a shows in schematic view lower part 101 and upper part 120 of the apparatus, in which lower part and upper part are in engagement with each other in the second position. Compared to the illustration of FIG. 1a, in the cross-sectional view the formed cavity 130 is shown as substantially circular, so that the cavity itself is substantially cylindrical. Contrary to the illustration of FIG. 1a, the upper part 120 is located in the receiving area 107 of the lower part. In the embodiment shown, the lower tube half shell 102 is oversized at the side edges 103a and 103b relative to the upper tube half shell 120 at the side edges 122a and 122b. When the upper part is lowered, the side edges 122a and 122b of the upper tube half-shell can thus penetrate with the chamfers 127a and 127b to the inside 104 of the lower tube half-shell 101. According to FIG. 2a, the side edges 122a and 122b thereby lie against the surface 105 of the lower tube half-shell 102. Hollow fiber membranes (not shown in FIG. 2a), which lie against the surface 105 of the lower tube half-shell 102, are wiped off via the chamfers 124a and 124b. Lowering the upper part compresses the array of hollow fiber membranes in the cavity 130. Alternatively, there may be minimal gaps of less than one hollow fiber membrane diameter between the side edges 122a and 122b and the surface 105 of the lower tube half-shell 102.

FIG. 2b shows a side view of the lower part and upper part in the second position corresponding to FIG. 2a.

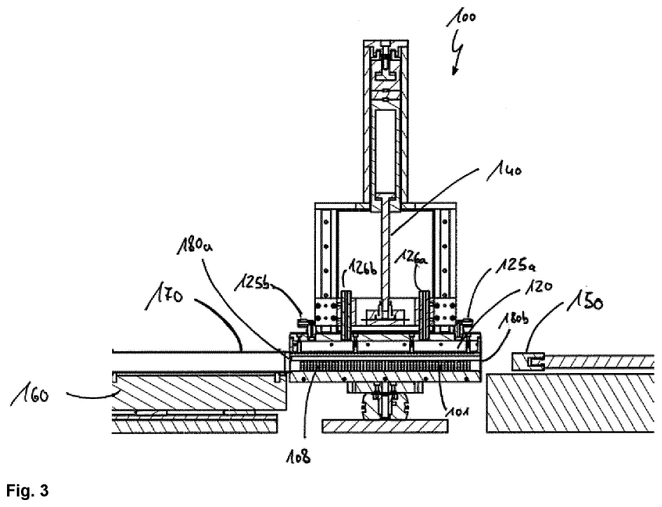

FIG. 3 shows, in a cross-sectional view, an illustration of an apparatus 100 having a lower part 101 and an upper part 120 in a second position in which the lower part 101 and the upper part 120 are in engagement with each other. In the cross-sectional view, the drillings 108 of the surface 105 of the lower tube half shell 102 are schematically shown. FIG. 3 shows a lifting apparatus 140 for moving the upper part 120 from a first position to a second position via the retaining elements 126a and 126b. Further schematically shown is an ejector 150, which is movable according to an embodiment corresponding to FIG. 3. Further shown in FIG. 3 is a receiving unit 160 which receives a cylindrical housing tube 170. The receiving unit is a movable component by means of which a housing tube, in particular a cylindrical housing tube, can be arranged in the second position shown adjacent to the lower and upper tube half shells on the face side.

### Example

With reference to the embodiments shown in FIGS. 1a to 3, the bundling of an array of hollow fiber membranes according to the invention will be explained. First, an array of hollow fiber membranes is introduced into the lower tube half-shell 102 in the first position of the lower part 101 and the upper part 120. The radius of the lower and upper tube half-shells (102, 121) is thereby dimensioned such that a predetermined number of hollow fiber membranes can be bundled into a hollow fiber membrane bundle. A hollow fiber membrane commonly used for hemodialysis is used. This has an outer diameter of 261 μm. The hollow fiber membrane used is textured, i.e. the hollow fiber membrane has a waveform known in the prior art with an amplitude of 0.41 mm and a wavelength of 7.5 mm. For the manufacturing of a hollow fiber membrane filter, 8448 of these hollow fiber membranes are inserted into the lower tube half-shell of the lower part of an apparatus according to the invention. The diameter of the concavely curved surface of the tube half-shell is 29 mm.

In a next step, the upper part 120 is displaced in the apparatus 100 towards the receiving area 107 of the lower part until the upper tube half-shell 121 and lower tube half-shell 102 enclose a cavity 130, which is substantially cylindrical according to the example explained here. The displacement of the upper part into the receiving area 107 of the lower part 101, occurs until the array of hollow fiber membranes in the cavity 130 forms a cylindrical bundle having a diameter of about 29 mm. In this compressed state, the hollow fiber membrane bundle has a packing density of 68.4%. In this context, packing density is understood as the ratio of the sum of all cross-sectional areas of the 8448 hollow fiber membranes to the cross-sectional area of the substantially cylindrical cavity 130. Subsequently, the hollow fiber membrane bundle is cut to a predetermined length for the construction of hollow fiber membrane filters using a cutting tool.

In a further step, air is supplied to the inner sides 104 and 123 of the lower and upper tube half-shells 102, 121 via gas ports 125a/b and 106a-c, respectively. A cylindrical housing tube is positioned via the receiving unit 160 adjacent the face of an opening side 180a of the cavity 130 formed by the upper and lower tube half-shells. An ejector 150 is positioned on the opposite opening side 180b. The ejector 150, which is movable in a longitudinal direction relative to the tube half-shells, is set in motion and pushes the hollow fiber membrane bundle out of the cavity 130 into the adjacent cylindrical housing tube 160. In a preferred embodiment, the cylindrical housing tube is the housing of a hollow fiber membrane filter. Alternatively, another housing tube may be used into which the hollow fiber membrane bundle is inserted so that the hollow fiber membrane bundles can be removed from the apparatus one piece at a time.

Accordingly, the cylindrical housing tube can be a housing of a hollow fiber membrane filter having an inner radius of 31 mm. The packing density of the hollow fiber membrane bundle in the cylindrical tube is then 59.9%.

Alternatively, a thin-walled metal tube can be used, and the hollow fiber membrane bundle can be inserted from the cavity 130 into the thin-walled metal tube. In this case, the thin-walled metal tube has an inner radius of 15 mm and an outer radius of 15.4 mm. The hollow fiber membrane bundle can thus be initially inserted into the thin-walled metal tube. For the further manufacture of a hollow fiber membrane filter, the thin-walled metal tube is inserted into the housing of a hollow fiber membrane filter with an inner radius of 15.5 mm. The metal tube is then pulled out of the housing of the hollow fiber membrane filter, with the hollow fiber membrane bundle being held against it and remaining in the housing of the hollow fiber membrane filter.

Housings with inserted hollow fiber membrane bundles are then fed to further process steps of the hollow fiber membrane filter production.

1. An apparatus for bundling hollow fiber membranes, comprising a lower part comprising a lower tube half-shell having two side edges and an inner side comprising a concavely curved surface for receiving an array of hollow fiber membranes, an upper part comprising an upper tube half-shell complementary to the lower tube half-shell and having two side edges and an inner side comprising a concavely curved surface wherein lower part and/or upper part are arranged movably relative to each other in the apparatus, and wherein the apparatus is configured such that the lower part and the upper part are positioned in a first position such that the lower tube half-shell is capable of receiving an array of hollow fiber membranes in the first position, and the lower part and upper part are positioned in a second position such that the lower tube half-shell and the upper tube half-shell enclose a cavity such that an array of hollow fiber membranes present in the cavity is capable of being bundled.

2. The apparatus according to claim 1, wherein the concavely curved surface of the lower tube half-shell describes a segment of a substantially cylindrical shape, and in that the concavely curved surface of the upper tube half-shell describes a segment of a substantially cylindrical shape, so that the concavely curved surfaces of the lower and the upper tube half-shells enclose a substantially cylindrical cavity in a second position of the lower part and the upper part.

3. The apparatus according to claim 1 wherein the lower tube half-shell is oversized relative to the upper tube half-shell in the region of the side edges of the lower tube half-shell, or in that the upper tube half-shell is oversized relative to the lower tube half-shell in the region of the side edges of the upper tube half-shell, the apparatus being configured such that
in the second position of lower part and upper part, the upper tube half-shell is in engagement with the lower tube half-shell, or in that in the second position of the lower part and the upper part, the lower tube half-shell is in engagement with the upper tube half-shell.

4. The apparatus according to claim 1, wherein the side edges of the upper tube half-shell and/or the lower tube half-shell are chamfered.

5. The apparatus according to claim 1, wherein the concavely curved surfaces of the lower and/or the upper tube half-shell comprise a plurality of drillings, and the apparatus is configured to flow air through the drillings onto the inner side of the lower and/or the upper tube half-shell.

6. The apparatus according to claim 5, wherein at least a part of the drillings in the lower and/or upper tube half-shell are aligned in a direction that is abutting at a coinciding angle of 10° to 80° or 20° to 70° or 30° to 60° to the central axis of the cylindrical cavity.

7. The apparatus according to claim 1, wherein the concavely curved surfaces of the lower and/or upper tube half-shells are provided with a coating.

8. The apparatus according to claim 1, wherein the apparatus comprises at least one movable cutting device for cutting the hollow fiber membrane bundle in the second position of the upper part and lower part to a predetermined measure of length.

9. The apparatus according to claim 1, wherein the apparatus comprises a receiving area for a housing tube, which is movably arranged in the apparatus relative to the lower part and/or to the upper part, and the apparatus is further configured such that in the second position of upper part and lower part a housing tube can be arranged adjoining the lower and upper tube half shell on the face side via a position of the receiving area.

10. The apparatus according to claim 9, wherein the apparatus comprises means for inserting the hollow fiber membrane bundle from the cavity formed by the upper and lower tube half-shells into a housing tube disposed end-to-end.

11. An arrangement comprising an apparatus according to claim 1, and a hollow fiber membrane bundle arranged in the cavity formed in the apparatus from lower and upper tube half-shells, wherein neither the hollow fiber bundle nor the apparatus comprises an envelope film.

12. The arrangement according to claim 11 further comprising a housing tube, wherein the diameter of the hollow fiber bundle located in the cavity is at least 2% smaller than the diameter of the housing tube.

13. The arrangement according to claim 12, wherein the one housing tube is arranged adjacent to the lower and upper tube half-shell at the end side and has a tapered middle part, and wherein the diameter of the hollow fiber bundle located in the cavity is at least 2% smaller than the diameter of the end of the housing tube disposed adjacent to the lower and upper tube half-shell at the end side.

14. A method of bundling hollow fiber membranes, comprising the steps of:
providing an apparatus according to claim 1,
placing an array of hollow fiber membranes in the lower tube half-shell in the first position of the upper part and lower part of the apparatus,
relatively moving the upper and lower parts to the second position so that the array of hollow fiber membranes is bundled into a hollow fiber membrane bundle in a cavity formed by the lower and upper tube half-shells.

15. The method according to claim 14, wherein the hollow fiber membrane bundle is lengthened to a predetermined length dimension in the second position of the upper part and the lower part by means of a cutting device, and wherein the cutting device is a hot cutting tool which melts the ends of the hollow fiber membranes during cutting to length and closes the lumens of the hollow fiber membranes.

16. The method according to claim 14, wherein a tube is positioned adjacent to the cavity formed by lower and upper tube half-shells and the hollow fiber membrane bundle is pushed into the adjacent tube.

17. The method according to claim 14, wherein the inner side of the lower and/or the upper tube half-shell is flowed with air through the plurality of drillings.

18. The method according to claim 14, wherein the array of hollow fiber membranes is not wrapped in an envelope film.

19. (canceled)

20. The arrangement according to claim 11 further comprising a housing tube, wherein the diameter of the hollow fiber bundle located in the cavity is at least 7% smaller than the diameter of the housing tube.

21. The arrangement according to claim 12, wherein the one housing tube is arranged adjacent to the lower and upper tube half-shell at the end side and has a tapered middle part, and wherein the diameter of the hollow fiber bundle located in the cavity is at least 7% smaller than the diameter of the end of the housing tube disposed adjacent to the lower and upper tube half-shell at the end side.
